(19) United States
(12) Patent Application Publication
(10) Pub. No.: US 2024/0237934 A1
(43) Pub. Date: Jul. 18, 2024

(54) WEARABLE DEVICE FOR ACQUIRING PLURAL ELECTROCARDIOGRAM LEAD SIGNALS

(71) Applicant: HEXACHECK INC., Daejeon (KR)

(72) Inventor: In-Duk HWANG, Sejong City (KR)

(73) Assignee: HEXACHECK INC., Daejeon (KR)

(21) Appl. No.: 18/010,201

(22) PCT Filed: Aug. 16, 2022
(86) PCT No.: PCT/KR2022/012211

§ 371 Date: Jan. 12, 2023

(30) Foreign Application Priority Data
Aug. 13, 2021 (KR) 10-2021-0107569

(51) Int. Cl. A61B 5/282; A61B 5/00; A61B 5/256; A61B 5/26; A61B 5/332

(52) U.S. Cl. CPC A61B 5/282; A61B 5/0006; A61B 5/0024; A61B 5/256; A61B 5/26; A61B 5/332; A61B 5/681; A61B 2560/0209; A61B 2560/0468

(57) ABSTRACT

The present invention relates to a wearable device for acquiring plural electrocardiogram lead signals, and more particularly, to a wearable device as a plurality of electrocardiogram measuring devices (measurement sensors) wearable by an individual, which is convenient to carry so as to be easily used regardless of time and place, and configured to acquire six electrocardiogram lead signals through two limb lead signals measured simultaneously.

### TECHNICAL FIELD

The present invention relates to a wearable device for acquiring plural electrocardiogram lead signals, and more particularly, to a wearable device as a plurality of electrocardiogram measuring devices (measurement sensors) wearable by an individual, which is convenient to carry so as to be easily used regardless of time and place and configured to acquire six electrocardiogram lead signals through two limb lead signals measured simultaneously.

The present invention, as a device for measuring a plurality of electrocardiograms, may be classified into A61B 5/04 class that includes detecting, measuring, or recording bioelectric signals of the body or parts thereof, based on the International Patent Classification (IPC).

### BACKGROUND ART

An electrocardiograph provides a waveform of an electrical signal, that is, an electrocardiogram (ECG), which can be easily obtained and contains very useful information in order to analyze the state of a patient's heart.

In other words, the electrocardiograph is a useful device that can conveniently diagnose the state of the patient's heart. The electrocardiograph may be classified into several types depending on the purpose of use. A 12-channel electrocardiograph using 10 wet electrodes is used as a standard electrocardiograph for hospitals to obtain as much information as possible. A Holter recorder and an event recorder, which can be used by a user while moving, have the following essential features. The features include having a small size, using a battery, and having a storage device for storing measured data and a communication device for transmitting the data.

Meanwhile, the event recorder enables the user to measure ECG on the spot when the user feels an abnormality in the heart while carrying the event recorder. Accordingly, the event recorder has a small size, is not provided with cables for connecting electrodes, and is provided with dry electrodes on a surface of the event recorder. Event recorders in the related art are mostly 1-channel, that is, 1-lead electrocardiographs for measuring one ECG signal by bringing both hands into contact with two electrodes, respectively.

The electrocardiogram measurement device pursued by the present invention is required to be convenient for individual use, required to provide accurate and sufficient electrocardiogram measurement values, and required to be small for easy portability. In order to be convenient for individual use, the claimed device is required to transmit data through wireless communication. In addition, the claimed device is required to be operated with a battery.

According to the present invention, two limb leads measured simultaneously are acquired in order to provide accurate and sufficient electrocardiogram measurement values. As described later, according to the present invention, four leads may be calculated and provided from measurement values obtained by simultaneously measuring the two limb leads. In general, regarding the electrocardiogram, the terms “channel” and “lead” are used interchangeably, and refer to one electrocardiogram signal or electrocardiogram voltage. Regarding the electrocardiogram, the word “simultaneously” is required to be used very carefully. Specifically, when lead II is sampled while a voltage of lead I is being sampled at a constant sampling period, it can be said that a measurement is simultaneously conducted only when each time point for sampling the lead II is within less than half of the sampling period from each time point for sampling the lead I. In addition, the word “measurement” is also required to be used carefully. The word “measurement” is required to be used only when a physical quantity is actually measured. In digital measurement, one measurement is required to substantially signify one AD conversion. As described later, in the electrocardiogram measurement, lead II can be calculated according to Kirchhoff voltage law by measuring leads I and leads III, for example. In this case, it is accurate when lead II is expressed as “calculated”, and when the expression “measured” is used, it may cause confusion.

One of the most difficult problems in the electrocardiogram measurement is removing power line interference included in an electrocardiogram signal. A driven right leg (DRL) scheme is well-known for removing the power line interference.

In addition, recently, an electrocardiograph mounted on a smart watch is being used very usefully. However, the electrocardiograph mounted on the smart watch provides only the electrocardiogram signal between both hands, that is, the lead I signal, and accordingly medical information may be provided insufficiently. Thus, there is a need for a device capable of providing a larger number of electrocardiogram signals.

### DISCLOSURE

### Technical Problem

The present invention has been made in view of the above problems and needs, and provides an electrocardiograph device, by using a watch equipped with an electrocardiograph, in which two limb lead signals measured simultaneously are acquired. It is very important in the medical field to measure two limb leads simultaneously. This is because it takes more time and is inconvenient to measure the two leads sequentially. In addition, this is because the two limb leads measured at different times may not correlate with each other and may cause confusion in an accurate and detailed determination of arrhythmia. More importantly, this is because the two limb lead signals are required to be measured simultaneously in order to acquire a total of 6 limb leads by calculating 4 additional limb leads as described later.

Since the electrocardiograph mounted on the smart watch measures the lead I signal, one of lead II or lead III is required to be measured and obtained in order to obtain a total of 6 limb leads by the method described below. Further, the method of measuring the one of lead II or lead III is required to be convenient for users. In addition, the structure of the device used when measuring the one of lead II or lead III and the arrangement of the electrodes are required to be convenient for users.

The present invention employs an electrocardiograph disposed in a watch band in order to solve the above problems and satisfy the needs.

However, the electrocardiograph disposed in the band employed in the present invention is required to wirelessly communicate with the electrocardiograph mounted on the watch in order to transmit the measured electrocardiogram signal to the electrocardiograph mounted on the watch. However, since a time delay inevitably occurs in wireless communication, the time delay is required to be compensated to obtain two electrocardiogram signals measured simultaneously. Thus, there is a problem that a time delay value generated during wireless communication is required to be obtained.

Meanwhile, one portable measuring device generally uses a battery and requires a mechanical power switch for controlling power consumption of the battery. However, the mechanical power switch increases the volume or area of the portable measuring device, causes the limit of miniaturization, and increases the possibility of failure.

### Technical Solution

The present invention has been made in view of the above problems and needs, and provides an electrocardiograph device, by using a watch equipped with an electrocardiograph, to acquire two limb leads measured simultaneously, and according to the embodiments, an additional mechanical switch may not be used or may be used as needed.

The wearable device as an electrocardiogram measuring device according to the present invention for achieving the above objects includes: a watch worn by a user on one wrist; one band coupled to the watch; a first electrocardiograph coupled to the one band and disposed at a position facing a bottom surface of the watch; and a second electrocardiograph included in the watch, wherein the first electrocardiograph includes a first electrode disposed on an inner surface of the band to come into contact with the one wrist of the user, and a second electrode disposed on an outer surface of the band to come into contact with a left knee or left ankle of the user, and the second electrocardiograph includes a third electrode disposed on the bottom surface of the watch to come into contact with the one wrist of the user, and a fourth electrode coming into contact with an opposite hand of the user.

In addition, the first electrocardiograph may measure a first electrocardiogram lead signal induced between the first and second electrodes, and transmit the measured first electrocardiogram lead signal to the second electrocardiograph by using a wireless communication mechanism, and the second electrocardiograph may measure a second electrocardiogram lead signal through the third and fourth electrodes, receive the first electrocardiogram lead signal by using the wireless communication mechanism, and compensate for a time delay generated in a wireless communication process based on the received first electrocardiogram lead signal, so that the first electrocardiogram lead signal and the second electrocardiogram lead signal become two electrocardiogram lead signals sampled at the same time (simultaneously or synchronously).

In addition, the wearable device may additionally calculate four electrocardiogram lead signals by using the two electrocardiogram lead signals sampled at the same time, thereby acquiring six limb lead signals including lead I, lead II, lead III, lead aVR, lead aVL, and lead aVF.

In addition, the first electrocardiograph may include one microcontroller for controlling the first electrocardiograph, and the microcontroller is operated in a sleep mode when the first electrocardiograph does not measure an electrocardiogram lead signal so as to power off an amplifier, an AD converter, and the wireless communication mechanism included in the first electrocardiograph, and, when switched to an active mode, power on the amplifier, the AD converter, and the wireless communication mechanism, amplify and AD-convert the first electrocardiogram lead signal, and perform wireless communication.

In addition, the first electrocardiograph may include one current sensor supplied with power, in which the current sensor may allow a current to flow through the user's body when the first electrode contacts the one wrist of the user and the second electrode contacts the left knee or the left ankle of the user, and generate an output signal when sensing the current, and the microcontroller may change a sleep mode to an active mode when receiving the output signal of the current sensor.

In addition, the wearable device according to the present invention may use a time delay value determined using the following processes (a) to (d).
(a) One output signal of one signal generator is commonly applied to the first electrocardiograph and the second electrocardiograph.
(b) The first electrocardiograph and the second electrocardiograph measure the output signal,
(c) The first electrocardiograph transmits the measured signal through the wireless communication mechanism, and the second electrocardiograph receives the transmitted signal,
(d) Two waveforms of the signal measured by the second electrocardiograph and the signal received by the second electrocardiograph are compared.

In addition, the band may be configured such that a length of the band may be formed longer than a length of the opposite band to accommodate the first electrocardiograph.

In addition, the wireless communication mechanism may be implemented in a Bluetooth Low Energy.

In addition, a method for acquiring a plurality of electrocardiogram leads using an electrocardiograph accommodated in a watch worn on one wrist and an electrocardiograph attached to a band of the watch according to the present invention includes: bringing a first electrode of the electrocardiograph attached to the band into contact with a wrist and bringing a second electrode into contact with a left leg or a left ankle; switching a microcontroller accommodated in the electrocardiograph attached to the band to an active mode; powering on, when the microcontroller is switched to the active mode, the amplifier, the AD converter, and the wireless communication mechanism; amplifying an electrocardiogram lead between the first electrode and the second electrode; converting the amplified analog signal into a digital signal; transmitting the first electrocardiogram lead data converted into the digital signal to an electrocardiograph accommodated in the watch by using the wireless communication mechanism; receiving, by the electrocardiograph accommodated in the watch, the transmitted first electrocardiogram lead data through the wireless communication mechanism; and making the first electrocardiogram lead data and second electrocardiogram lead data which is measured through electrodes attached to the watch by compensating for a time delay to the received first electrocardiogram lead data, which is generated during a wireless communication process and the like, as a set of two electrocardiogram lead data sampled at the same time.

In addition, the method for acquiring a plurality of electrocardiogram lead signals may further include checking a presence of a flow of a current in the current sensor in order to determine whether to finish measuring the electrocardiogram after the microcontroller accommodated in the electrocardiograph attached to the band measures the electrocardiogram for a certain period of time.

In addition, one embodiment of the present invention provides a wearable device including: one watch electrocardiograph for measuring lead I; and one inferior lead electrocardiograph for measuring one of lead II or lead III according to an installed position.

In addition, according to the present invention, a difference between time points for sampling the two electrocardiogram lead signals may be less than a sampling period to obtain the two electrocardiogram lead signals sampled in the same time band (simultaneously or synchronously).

In addition, in order to achieve the above-mentioned objects, the present invention proposes a wearable device including: one watch electrocardiograph installed in one watch body to measure lead I; and one inferior lead electrocardiograph for measuring one of lead II or lead III according to an installed position, wherein the watch electrocardiograph wirelessly transmits a command for starting electrocardiogram measurement (electrocardiogram measurement start command) to the one inferior lead electrocardiograph, the watch electrocardiograph measures lead I, the one inferior lead electrocardiograph wirelessly receiving the electrocardiogram measurement start command measures one of lead II or lead III, and the one inferior lead electrocardiograph wirelessly transmits the measured one of leads II or lead III to the watch electrocardiograph, and then the watch electrocardiograph wirelessly receives the transmitted one of the lead II or lead III, so that two electrocardiogram lead signals measured in the same time band are acquired; and four electrocardiogram lead signals are additionally calculated by using the two electrocardiogram lead signals measured in the same time band, so that six limb lead signals including Lead I, Lead II, Lead III, Lead aVR, Lead aVL, and Lead aVF are acquired.

The one inferior lead electrocardiograph for measuring one of lead II or lead III may include one electrode coupled to one band coupled to the one watch body, disposed at a position facing a bottom surface of the watch body, and disposed on an inner surface of the band to contact the one wrist of the user, and one electrode disposed on an outer surface of the band to come into contact with a left knee or left ankle of the user.

In one embodiment, the one inferior lead electrocardiograph for measuring one of lead II or lead III, may have a ring shape worn on one finger.

In one embodiment, the one inferior lead electrocardiograph for measuring one of lead II or lead III, may have a patch or chest-band shape, and may include electrodes in contact with the chest.

In addition, according to the present invention, the two electrocardiogram lead signals measured in the same time band may have the same frequency response characteristics.

In addition, according to the present invention, the two electrocardiogram lead signals measured in the same time band may have the same gain characteristics.

In addition, according to the present invention, the two electrocardiogram lead signals measured in the same time band may have a maximum amplitude error within +/−5%.

In addition, according to the present invention, the two electrocardiogram lead signals measured in the same time band may be sampled at the same sampling rate.

In addition, a wireless type for communicating between the watch electrocardiograph and one inferior lead electrocardiograph may include Bluetooth Low Energy.

In addition, after the connection of the Bluetooth Low Energy is established, the one inferior lead electrocardiograph, may sample the electrocardiogram lead signal during one connection interval, and transmit the sampled data during one connection event following the sampling.

The connection interval may be an integral multiple of a sampling period when the one inferior lead electrocardiograph samples one electrocardiogram lead signal.

In addition, according to the present invention, the watch electrocardiograph and the one inferior lead electrocardiograph may sample each electrocardiogram lead signal at the same time by sampling each electrocardiogram lead signal after the same amount of time has elapsed from the connection event.

In addition, according to the present invention, an operation of additionally calculating the four electrocardiogram lead signals, and an operation of displaying six limb induction signals may be performed on a smartphone.

In addition, according to the present invention, the one electrocardiograph generates the electrocardiogram measurement start command, after a photoplethysmograph mounted together with one electrocardiograph detects an abnormality in cardiac activity and generates an alarm.

In addition, according to the present invention, the inferior lead electrocardiograph or the watch electrocardiograph generates the electrocardiogram measurement start command, after a current sensor detects that the user has brought the user's body into contact with two electrodes of the inferior lead electrocardiograph to measure the electrocardiogram and generates an output.

### Advantageous Effects

The wearable device according to the present invention may be convenient to carry, may be easily used regardless of time and place, and may acquire six electrocardiogram lead signals. Thus, it can be very useful for health care.

### DESCRIPTION OF DRAWINGS

FIG. 1 is a perspective view of a wearable device when viewed from one direction according to the present invention.

FIG. 2 is a perspective view of the wearable device when viewed from a different direction according to the present invention.

FIG. 3 is a block diagram of a second electrocardiograph according to the present invention.

FIG. 4 is a perspective view of a ring-shaped electrocardiograph used in the present invention.

FIG. 5 is a view showing a state in which a patch electrocardiograph used in the present invention is attached to a user's chest.

FIG. 6 is a view showing a state in which a chest band electrocardiograph used in the present invention is worn on the user's chest.

FIG. 7 is a diagram showing the operations of sampling electrocardiogram lead signals and transmitting and receiving the sampled data, respectively, in a state in which two electrocardiographs are connected via Bluetooth Low Energy according to the present invention.

### BEST MODE

As a best mode, the present invention provides a wearable device including: one watch electrocardiograph installed in one watch body to measure lead I; and one inferior lead electrocardiograph for measuring one of lead II or lead III according to an installed position, wherein
the watch electrocardiograph wirelessly transmits a command for starting electrocardiogram measurement to the one inferior lead electrocardiograph,
the watch electrocardiograph measures lead I,
the one inferior lead electrocardiograph wirelessly receiving the electrocardiogram measurement start command measures one of lead II or lead III, and
the one inferior lead electrocardiograph wirelessly transmits the measured one of lead II or lead III to the watch electrocardiograph, and then
the watch electrocardiograph wirelessly receives the transmitted one of the lead II or lead III, so that
two electrocardiogram lead signals measured in the same time band are acquired; and
four electrocardiogram lead signals are additionally calculated by using the two electrocardiogram lead signals measured in the same time band, so that six limb lead signals including Lead I, Lead II, Lead III, Lead aVR, Lead aVL, and Lead aVF are acquired.

### MODE FOR INVENTION

Hereinafter, a wearable device for acquiring plural electrocardiogram lead signals according to the present invention will be described in detail below with reference to the accompanying drawings. The drawings disclosed below are provided as examples to enable those skilled in the art to sufficiently understand the idea of the present invention. Therefore, the present invention is not limited to the drawings presented below and may be embodied in other forms. In addition, like reference numerals indicate like elements throughout the specification.

Unless otherwise defined, the technical terms and scientific terms used herein have the meaning commonly understood by a person having ordinary skill in the art, and the description of well-known functions and configurations that may unnecessarily obscure the subject matter of the present invention will be omitted in the following descriptions and accompanying drawings.

Prior to describing the present invention, it will be explained that if two limb lead signals are measured, four leads can be calculated and additionally obtained as described below. The above measurement method is a method provided according to the present invention to obtain six electrocardiogram lead signals in the most convenient manner. The principle of the present invention is as follows.

The conventional 12-lead ECG is described in, for example, [ANSI/AAMI/IEC 60601-2-25: 2011, Medical electrical equipment-part 2-25: Particular requirements for the basic safety and essential performance of electrocardiographs]. In the traditional 12-lead ECG, three limb leads are defined as follows. Lead I=LA−RA, Lead II=LL−RA, and Lead III=LL−LA. In the above equations, RA, LA, and LL refer to voltages of the right arm, left arm, and left leg, respectively, or the body parts close to the limbs. One limb lead may be obtained from the other two limb leads based on the above relationships. For example, Lead III=Lead II−Lead I. Three augmented limb leads are defined as follows. aVR=RA−(LA+LL)/2, aVL=LA−(RA+LL)/2, and aVF=LL−(RA+LA)/2. Accordingly, the three augmented limb leads may be obtained from two limb leads. For example, aVR may be obtained from “aVR=−(I+II)/2”. Accordingly, when two limb leads are measured, the remaining four leads may be calculated and obtained.

Hereinafter, the embodiments according to the present invention will be described with reference to the accompanying drawings.

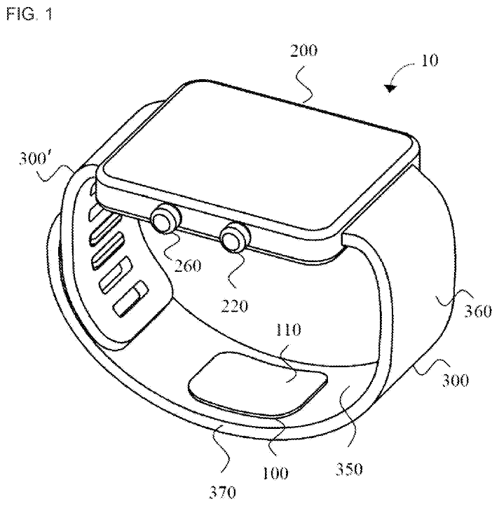

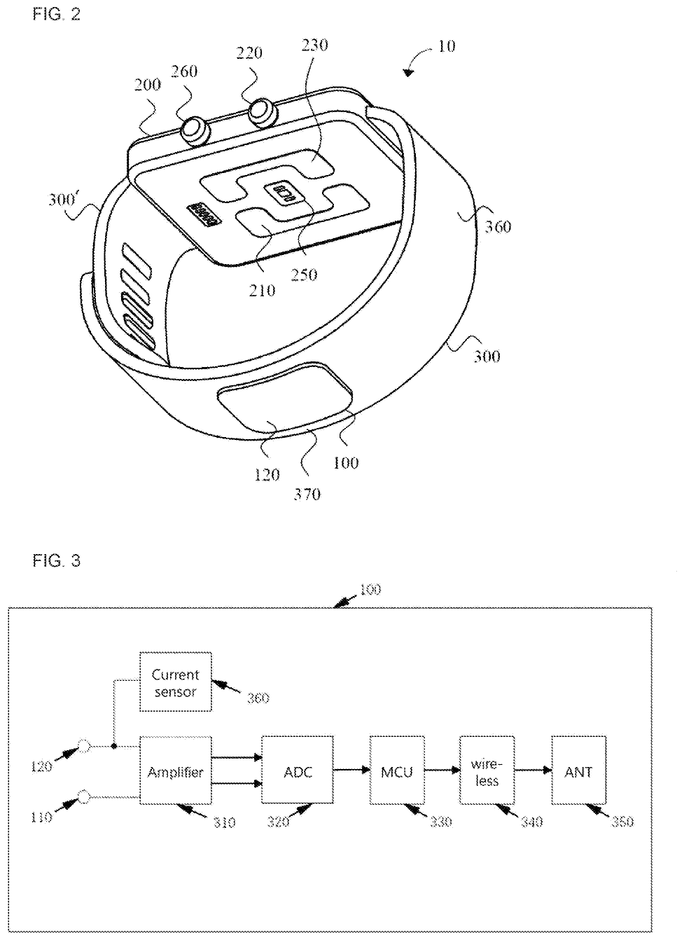

FIG. 1 is a perspective view of a wearable device when viewed from one direction according to the present invention. FIG. 2 is a perspective view of the wearable device when viewed from a different direction according to the present invention. The structure of the wearable device according to the present invention and the arrangement of used electrodes will be described with reference to FIGS. 1 and 2. The wearable device according to the present invention includes: a watch 200 worn by a user on one wrist, a band 300 coupled to the watch 200, a first electrocardiograph 100 coupled to the one band 300 and disposed at a position 370 facing a bottom surface of the watch 200, and a second electrocardiograph 200 included in the watch 200. According to the present invention, the band 300 is required to be longer than the band 300′ in order to dispose the first electrocardiograph 100 at the position 370 facing the bottom surface of the watch 200 (FIG. 1). According to the present invention, the watch may include other components 250 and 260 irrelevant to the second electrocardiograph. However, in the description of the present invention using FIGS. 1 and 2, the watch and the second electrocardiograph are denoted by the same reference numeral 200 for convenience.

In FIG. 1, the first electrocardiograph 100 includes a first electrode 110 disposed on an inner surface 350 of the band 300, and the second electrocardiograph 200 includes a fourth electrode 220 capable of coming into contact with a hand opposite to the hand on which the watch 200 is worn. In FIG. 2, the first electrocardiograph 100 includes a second electrode 120 disposed on an outer surface 360 of the band 300, and the second electrocardiograph 200 includes a third electrode 210 coming into contact with the wrist on which the watch 200 is worn.

In the embodiment, the first electrocardiograph 100 measures a first electrocardiogram lead signal induced between the first electrode 110 and the second electrode 120. In the case that the watch 200 is worn on the left wrist, the first electrocardiogram lead signal measured when the second electrode 120 comes into contact with the left knee or left ankle of the user is lead III.

In the embodiment, the second electrocardiograph 200 measures a second electrocardiogram lead signal induced between the third electrode 210 and the fourth electrode 220. In the case that the watch 200 is worn on the left wrist, the second electrocardiogram lead signal measured when the fourth electrode 220 comes into contact with a finger of the user's right hand is lead I.

The first electrocardiograph 100 and the second electrocardiograph 200 according to the present invention, are devices independent from each other, and are not wire-connected to each other. Thus, the first electrocardiograph 100 and the second electrocardiograph 200 according to the present invention, are connected to each other only by wireless communication.

In the embodiment, the first electrocardiograph 100 transmits the measured first electrocardiogram lead signal to the second electrocardiograph 200 through the wireless communication mechanism. The second electrocardiograph 200 receives the first electrocardiogram lead signal through the wireless communication mechanism.

According to the present invention, the first electrocardiograph 100 and the second electrocardiograph 200 are supplied with power from separate batteries, respectively. In FIGS. 1 and 2, it is important that the first electrocardiograph 100 may not include any mechanical switch. In the first electrocardiograph 100 as further described in FIG. 3, when a current flows between the first electrode 110 and the second electrode 120, a microcontroller accommodated in the first electrocardiograph 100 is switched to an active mode to power on devices inside the first electrocardiograph 100. When the electrocardiogram is not measured, the microcontroller turns off the devices inside the first electrocardiograph 100 and enters a sleep mode in order to prevent power consumption of the battery inside the first electrocardiograph 100.

FIG. 3 is a block diagram showing the internal structure of the first electrocardiograph 100. The electrocardiogram lead signal is inputted to the first electrode 110 and the second electrode 120. The amplifier 310 amplifies the inputted electrocardiogram lead signal. The AD converter 320 converts an input analog signal into a digital signal. The microcontroller 330 receives the AD-converted electrocardiogram lead signal and transmits the received AD-converted electrocardiogram lead signal through a wireless communication mechanism 340 and an antenna 350.

The current sensor 360 is always supplied with power from a built-in battery. When the left knee or left foot comes into contact with the second electrode 120 while the first electrode 110 is in contact with the wrist, the current sensor 360 allows a current to flow between the first electrode 110 and the second electrode 120 and switches the microcontroller 330 in the sleep mode to the active mode. Then, the microcontroller 330 powers on the wireless communication mechanism 340 and communicates with the second electrocardiograph 200 to check whether the second electrocardiograph 200 wants to measure an electrocardiogram. When the second electrocardiograph 200 wants to measure the electrocardiogram, the amplifier 310 and the AD converter 320 are powered on and the electrocardiogram is measured.

After the electrocardiogram is measured for a predetermined period of time, the state of the current sensor 360 is checked to determine whether to terminate the electrocardiogram measurement. In general, the electrocardiogram measurement is performed for about 30 seconds. The user may confirm that 30 seconds have elapsed through a display of the watch and stop contact with the electrocardiogram electrode. However, when the user wants to measure continuously for longer than 30 seconds, the user may just keep the electrodes in contact. When the flow of current is not detected by the current sensor 360, the microcontroller 330 turns off the amplifier 310 and the AD converter 320 and the microcontroller 330 enters sleep mode. Although the AD converter 320 has been described as a device separate from the microcontroller 330, the AD converter 320 may be built into the microcontroller 330.

The second electrocardiograph 200, via wireless communication, receives the first electrocardiogram lead signal transmitted by the first electrocardiograph 100. In this case, a time delay subject to the wireless communication protocol occurs for a predetermined time. In order to calculate a third electrocardiogram lead signal by applying Kirchhoff's Law using two electrocardiogram lead signals, the two electrocardiogram lead signals are required to be measured at the same time (synchronously). The expression “the two signals are measured at the same time” signifies that the difference between the two sampled time points is required to be less than a sampling period for sampling an analog signal into a digital signal. In general, the sampling period is about 3 ms in the electrocardiogram signal measurement. Therefore, when a time delay more than or equal to 1 ms occurs in wireless communication, the time delay is required to be compensated for.

A wireless communication suitable for the present invention is a Bluetooth Low Energy (BLE) having short range and low power characteristics. In order to find out the time delay occurring in the BLE, the following methods may be used.
(a) One output signal outputted from one signal generator is commonly applied to the first electrocardiograph and the second electrocardiograph.
(b) The first electrocardiograph and the second electrocardiograph measure the output signal.
(c) The first electrocardiograph transmits the measured signal through the wireless communication mechanism, and the second electrocardiograph receives the transmitted signal.
(d) Two waveforms of the signal measured by the second electrocardiograph and the signal received by the second electrocardiograph are compared.

A waveform of one output signal outputted from the one signal generator may be, for example, a triangular wave. In order to accurately check the time delay, the first electrocardiograph and the second electrocardiograph may measure the output signal by using a sampling period shorter than the sampling period used for electrocardiogram measurement.

the wearable device according to the present invention as described above may be convenient to carry, may be easily used regardless of time and place, and may acquire six electrocardiogram lead signals. Thus, it can be very useful for health care.

As described above, the first embodiment, which is configured to acquire six limb lead signals by using two electrocardiographs for each measuring one electrocardiogram lead, has been described. Hereinafter, a new embodiment will be described. In order to describe the new embodiment described later and the first embodiment described above as the invention having a unified idea, more appropriate terms and names may be used instead of the terms and names used in the first embodiment.

In the first embodiment, the second electrocardiograph 200 is installed on the watch body. This has been previously described with reference to FIG. 1. In addition, it has been described that the second electrocardiogram lead signal measured by the second electrocardiograph 200 is the electrocardiogram lead signal between both hands, that is, lead I. In addition, previously, the watch and the second electrocardiograph 200 have been denoted by the same reference numeral 200 for convenience. Accordingly, the name of the watch electrocardiograph 200 may be used instead of the name of the second electrocardiograph 200 for convenience. The watch electrocardiograph 200 measures lead I.

In the first embodiment, it has been described that the first electrocardiograph measures lead III when the watch is worn on the left wrist. Meanwhile, when the watch is worn on the right wrist, the first electrocardiograph measures lead II. In the art or literature on electrocardiography, lead II, lead III, and aVF are classified as inferior leads. Accordingly, the first electrocardiograph described in the first embodiment may also be referred to as an inferior lead electrocardiograph. When the name is used, the first embodiment may be expressed as follows. In other words, according to the present invention, the wearable device for acquiring six limb lead signals may be described as follows, as an electrocardiogram measuring device (measurement sensor).

A wearable device including one watch electrocardiograph 200 installed in one watch body to measure lead I; and one inferior lead electrocardiograph 100 for measuring one of lead II or lead III according to an installed position, wherein
the watch electrocardiograph 200 wirelessly transmits a command for starting electrocardiogram measurement to the one inferior lead electrocardiograph 100,
the watch electrocardiograph 200 measures lead I, and
the one inferior lead electrocardiograph 100 wirelessly receiving the electrocardiogram measurement start command measures one of lead II or lead III.

When the one inferior lead electrocardiograph 100 wirelessly transmits the measured one of leads II or lead III to the watch electrocardiograph 200,
the watch electrocardiograph 200 wirelessly receives the transmitted one of the lead II or lead III, so that
two electrocardiogram lead signals measured in the same time band are acquired; and
four electrocardiogram lead signals are additionally calculated by using the two electrocardiogram lead signals measured in the same time band, so that six limb lead signals including Lead I, Lead II, Lead III, Lead aVR, Lead aVL, and Lead aVF are acquired.

When the present invention is described as above, the first electrocardiograph 100 of the first embodiment may be described as follows.

The one inferior lead electrocardiograph (first electrocardiograph) 100 for measuring one of lead II or lead III includes
one electrode (first electrode) 110 coupled to one band 300 coupled to the one watch body 200, disposed at a position facing the bottom surface of the watch body, and disposed on an inner surface 350 of the band to contact the one wrist of the user, and
one electrode (second electrode) 120 disposed on the outer surface 360 of the band to come into contact with a left knee or left ankle of the user.

Hereinafter, a second embodiment will be described. In the first embodiment, the inferior lead electrocardiograph 100 is installed in the band 300 coupled to the watch. However, it is not necessarily required. In the second embodiment, the inferior lead electrocardiograph 100 has a ring shape 400 worn on one finger. Also in the second embodiment, the inferior lead electrocardiograph 100 measures one of Lead II or Lead III. Also in the second embodiment, the watch electrocardiograph 200 measures lead I as in the first embodiment.

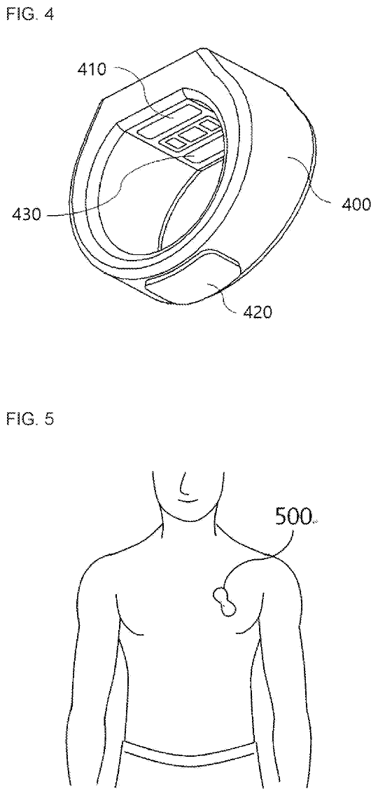

FIG. 4 shows a ring-shaped inferior lead electrocardiograph 400. The ring-shaped inferior lead electrocardiograph 400 includes at least one electrode 410 at an inner side of the ring, and one electrode 420 at a lower outer side of the ring. When the ring-shaped inferior lead electrocardiograph 400 is worn on the left hand and the outer electrode 420 comes into contact with the left leg, the ring-shaped inferior lead electrocardiograph 400 measures lead III. When the ring-shaped inferior lead electrocardiograph 400 is worn on the right hand and the outer electrode 420 comes into contact with the left leg, the ring-shaped inferior lead electrocardiograph 400 measures lead II. In FIG. 4, the at least one electrode 410 at the inner side of the ring has been expressed as being installed at a position spaced away from the outer electrode 420 for convenience, however, may be installed close to the outer electrode 420. In addition, the inferior lead electrocardiograph 400 may include a driven right leg electrode 430.

Hereinafter, a third embodiment will be described. In the third embodiment, the inferior lead electrocardiograph is a patch-shaped inferior lead electrocardiograph (patch electrocardiograph) 500 or a chest band-shaped inferior lead electrocardiograph (chest band electrocardiograph) 600. In the third embodiment, the patch or chest band-shaped inferior lead electrocardiograph 500 or 600 comes into contact with the chest to measure pseudo(quasi) lead II. Originally, lead II refers to an electrocardiogram signal induced between the right hand and the left leg. However, when the electrocardiograph is attached to an appropriate chest portion, an electrocardiogram signal almost similar to Lead II may be acquired, and this signal is called pseudo(quasi) lead II. Therefore, in order to measure pseudo lead II, it is necessary to carefully select a contact portion of the chest for the patch or chest band-shaped inferior lead electrocardiographs 500 or 600 to contact. In the third embodiment, the watch electrocardiograph 200 also measures lead I as in the first embodiment.

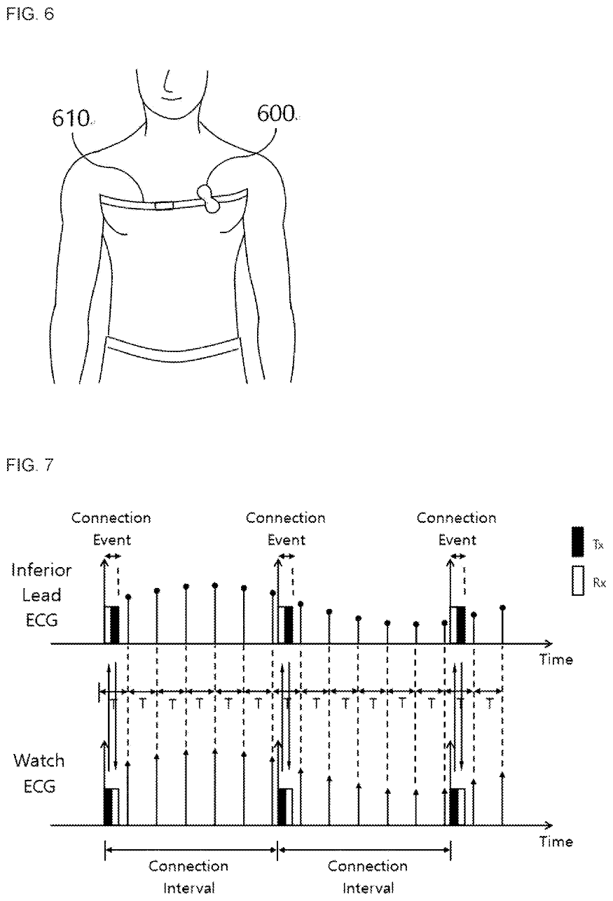

FIG. 5 shows the patch electrocardiograph 500 attached to the chest. The patch electrocardiograph 500 may be attached to the chest for about 2 weeks to continuously measure the electrocardiogram. FIG. 6 shows a chest band electrocardiograph 600 worn on the chest. The chest band electrocardiograph 600 is installed on an elastic band 610. Due to the use of dry electrodes, the chest band electrocardiograph 600 may be worn easily and used for a long period of time. A conventional chest band electrocardiograph 600 may obtain an electrocardiogram signal other than pseudo lead II. However, the chest band electrocardiograph 600 used in the present invention can obtain pseudo lead II and calculate another lead by using the obtained pseudo lead II. The patch electrocardiograph 500 or the chest band electrocardiograph 600 may measure one or two chest leads, such as V1, V2, V3, V4, V5, and V6, as needed.

The second and third embodiments have been described. The above-described contents of the first embodiment may also be applied to the second and third embodiments. In addition, the contents described hereinafter may be applied to all embodiments. According to the present invention, the expression “measuring in the same time band” signifies that the start time and the end time of two electrocardiogram measurements are the same. One measurement may, according to the context, signify one AD-conversion for an electrocardiogram lead signal, that is, one sampling.

One of the objects of the present invention is to additionally calculate four electrocardiogram lead signals by using two electrocardiogram lead signals measured by two electrocardiographs (the watch electrocardiograph and the inferior lead electrocardiograph) that communicate only wirelessly. Hereinafter, the conditions required for achieving the above objectives and devices and methods for satisfying the conditions will be described.

First, the equations for the generally known six limb electrocardiogram leads are summarized as follows. The following equations 1 to 6 are the equation for 6 limb leads among equations for the standard 12 leads described in the international medical device standard ANSI/AAMI/IEC 60601-2-25: 2011, Medical electrical equipment-part 2-25: Particular requirements for the basic safety and essential performance of electrocardiographs. RA, LA, and LL refer to voltages measured by the electrocardiograph at the right arm, left arm, and left leg, respectively, or the body parts close to the limbs.

$$I = LA - RA \qquad ( Equation 1 )$$

$$II = LL - RA \qquad ( Equation 2 )$$

$$III = LL - LA \qquad ( Equation 3 )$$

$$aVR = RA - ( LA + LL ) / 2 \qquad ( Equation 4 )$$

$$aVL = LA - ( RA + LL ) / 2 \qquad ( Equation 5 )$$

$$aVF = LL - ( RA + LA ) / 2 \qquad ( Equation 6 )$$

According to the present invention, as described below, it is very inventive to additionally calculate the four electrocardiogram lead signals by using the two electrocardiogram lead signals measured by the two electrocardiographs, respectively. The principles of the present invention to be described hereinafter have already been briefly described above in the section described with respect to FIG. 1.

According to the present invention, when two electrocardiographs measure lead I and lead II, respectively, the four leads are obtained using the following equations.

$$III = - I + II \qquad ( Equation 7 )$$

$$aVR = - ( I + II ) / 2 \qquad ( Equation 8 )$$

$$aVL = I - II / 2 \qquad ( Equation 9 )$$

$$aVF = - I / 2 + II \qquad ( Equation 10 )$$

In the present invention, it is very inventive to use Equations 7 to 10. Thomson et al., disclose Equations 8 to 10 (U. S. Patent Application Publication, Pub. No.: US2015/0018660 A1, Pub. Date: Jan. 15, 2015, application Ser. No. 14/328,962, Claim 28). However, Thomson et al., measure three voltages of RA, LA, and LL in order to use the above three equations. Whereas, in the present invention, two electrocardiogram lead signals, that is, two electrocardiogram voltages are measured. Therefore, the present invention is more effective than that of Thomson et al. In addition, Thomson et al., use Equation 3, that is, III=LL−LA. In other words, Equation 7 is not used (it has been described above that Thomson et al., use only Equations 8 to 10). In addition, the present invention discloses the following Equations 11 to 14 in addition to Equations 7 to 10. Therefore, the present invention is different from that of Thomson et al. In addition, Thomson et al., use one electrocardiograph. Whereas, in the present invention, two electrocardiographs connected only wirelessly are used. The present invention may be more effective and more inventive, since two leads are measured by using the two electrocardiographs connected only wirelessly and six limb leads are obtained.

According to the present invention, when two electrocardiographs measure lead I and lead III, respectively, the four leads are obtained using the following equations.

$$II = I + III \qquad ( Equation 11 )$$

$$aVR = - I - III / 2 \qquad ( Equation 12 )$$

$$aVL = ( I - III ) / 2 \qquad ( Equation 13 )$$

$$aVF = I / 2 + III \qquad ( Equation 14 )$$

There are several points to be noted to implement the present invention. Each term of Equation 11 as a function of time will be expressed as follows.

$$Lead II ( to + nT ) = Lead I ( to + nT ) + Lead III ( to + nT ) \qquad ( Equation 15 )$$

Equation 15 signifies that the two measured leads are required to be sampled at the same time in order to obtain other leads from the two measured leads. In Equation 15, T represents the sampling period, and n represents the sampling number. It is assumed that the electrocardiogram measurement start command occurred at t=0. Then, ‘to’ represents the elapsed time until the first (n=0) sampling is performed (t=to). When the total sampling number is N+1, NT represents the measured total time. In one embodiment, when the sampling rate is 300 sps (samples/second), T is 3.333 ms. When measured for 30 seconds, N is 30 s/3.333 ms=9,000.

Equation 15 represents that the two electrocardiogram lead signals, that is, Lead I and Lead III, are sampled at the same sampling rate. Therefore, in order to use the equations in the present invention, the two electrocardiographs are required to sample electrocardiogram lead signals at the same sampling rate, respectively. When the sampling rate is different, the sampling rates may be converted to be the same by using interpolation. However, it is much more effective to use the same sampling rate.

Equation 15 expresses the ideal case and may be expressed as follows in an actual situation.

$$Lead II ( to + nT ) = Lead I ( to + nT ) + Lead III ( to + nT + del ) \qquad ( Equation 16 )$$

Wherein, ‘del’ is a time delay. The time delay del may occur because it is difficult to accurately know the transmitting and receiving times during the wireless communication process performed by the two electrocardiographs. In addition, del may occur due to differences in the operation of the wireless communication mechanism 340, the microcontroller 330, and the AD converter 320 of the two electrocardiographs. As a result, the time delay del represents a difference between time points of sampling the two electrocardiogram lead signals, that is, the delayed time. The time delay occurring during a wireless communication process may cause a difference in the sampling time points.

According to the present invention, in order to use equations 7 to 10 or equations 11 to 14, the difference del between time points of sampling the two electrocardiogram lead signals is required to be smaller than the sampling period T. Preferably, the difference del between time points of sampling the two electrocardiogram lead signals is required to be smaller than T/2. The present invention aims to obtain two electrocardiogram lead signals for using equations expressed in the form of Equation 15.

Hereinafter, according to the present invention, in order to use equations 7 to 10 or equations 11 to 14, additional conditions that are required to be satisfied by the two electrocardiographs used in the present invention or the two electrocardiogram lead signals measured by the two electrocardiographs will be described.

The wearable device according to the present invention is a medical device. Each of the two electrocardiographs used for implementing the present invention is required to conform to medical device certification standards. The applicable international standard is ANSI/AAMI/IEC 60601-2-47: 2012, Medical electrical equipment-part 2-47: Particular requirements for the basic safety and essential performance of ambulatory electrocardiographic systems.

In order to implement the present invention, the following conditions are required: The two electrocardiographs used in the present invention are required to have the same gain. When two electrocardiogram lead signals measured by two electrocardiographs having different gains are applied to any of the above equations, unsuitable results may be obtained. Herein, the gain includes the gain of the amplifier used in the electrocardiograph, and signifies the final gain obtained subject to performing digital signal processing after AD conversion. The digital signal processing may not be performed in the electrocardiograph having been performed the AD conversion, and may be performed on another electrocardiograph or a smartphone. In addition, the expression “same” signifies that a size of the difference is less than the tolerable range. Based on the international standard, the maximum amplitude error is required within 10% for the accuracy of the gain.

In order to implement the present invention, the following conditions are required: The accuracy of the gains of the two electrocardiographs used in the present invention is necessarily superior to the accuracy of the gain required by the international standard. For example, the maximum amplitude error is required to be within +/−5%. Otherwise, the accuracy of the lead calculated when Equations 7 to 14 are applied may have the maximum amplitude error of 10% or more. This will be described in Table 1 with a case.

Table 1 shows an error analysis case when aVF is obtained by Equation 10.

TABLE 1
Example error analysis in case of obtaining
aVF according to Eq. 10. (aVF = −I/2 + II)
When input values are I = 0.60 mV, and II = 1.00 mV

| | Measured | Obtained | (obtained |
|---|---|---|---|
| | values(mV) | aVF (mV) | aVF)/0.70 |
| Ideal case | I = 0.60 | avF = 0.70 | 100% |
| | II = 1.00 | | |
| Error of 10% | I = 0.54 | aVF = 0.83 | 119% |
| | II = 1.10 | | |
| Error of 5% | I = 0.57 | aVF = 0.765 | 109% |
| | II = 1.05 | | |

The case in Table 1 shows that, Lead I is measured as 0.54 mV and Lead II is measured as 1.10 mV when applying 0.60 mV to lead I and 1.00 mV to lead II as test signals. In this case, the accuracy of the measurement is within the tolerance by the international standard. However, aVF is calculated and resulted in 0.83 mV based on the above measured values by using Equation 10, and this is 119% of 0.70 mV which is a value without an error. In this case, the error occurs by 19%. This exceeds the 10% tolerance of the standard. When it is assumed that the tolerance of measurement error for Lead I and Lead II is 5%, aVF is 0.765 mV based on Equation 10. In other words, an error occurs by 9%, and the international standard can be satisfied. Accordingly, in order to implement the present invention, the two electrocardiographs are required to have the measurement accuracy superior to the international standard.

In order to implement the present invention, the following conditions are required: The two electrocardiographs used in the present invention are required to have the same frequency response characteristics. Based on the international standard, the frequency response requirement during testing with a sine wave is as follows: The amplitude response in the frequency range 0.67 Hz to 40 Hz is required to be within 140% and 70% of the amplitude response at 5 Hz.

In order to implement the present invention, the following conditions are required: The two electrocardiographs used in the present invention are required to have the frequency response characteristics superior to the requirement of the international standards. The reason is the same as the reason for the need for superior gain accuracy described above. For example, the amplitude response in the frequency range 0.67 Hz to 40 Hz is required to be within 120% and 85% of the amplitude response at 5 Hz.

The two electrocardiographs used in the present invention are connected to each other only through wireless communication. This is because it is inconvenient to connect the two electrocardiographs used in the present invention by wire or each electrocardiograph manufacturer may manufacture the electrocardiograph for measuring only one electrocardiogram lead. It has been described above that the suitable wireless communication used in the present invention is Bluetooth Low Energy (BLE). The Bluetooth Low Energy is suitable for reducing the power consumption of a battery accommodated in the wearable device in situations where there is relatively little data to be transmitted and received and high-speed transmission and reception are not necessary as in the present invention.

FIG. 7 shows an embodiment in which the watch electrocardiograph 200 and the inferior lead electrocardiographs 100, 400, 500, and 600 communicate in the Bluetooth Low Energy according to the present invention. In FIG. 7, the operation of the watch electrocardiograph 200 is indicated below, and the operation of the inferior lead electrocardiographs 100, 400, 500, and 600 is indicated above as time passes. In this embodiment, the two electrocardiographs, for example, have the same sampling rate of 300 sps and the sampling is performed with a period T of 3.33 ms. After the connection between master and slave is established in Bluetooth Low Energy, a connection event is generated at every predetermined connection interval. Transmission and reception are performed in one connection event. In the embodiment of FIG. 7, the inferior lead electrocardiographs 100, 400, 500, and 600 perform 6 samplings during the connection interval of 20 ms, thereby transmitting 6 sampled data in one connection event followed by samplings. The watch electrocardiograph 200 performs the sampling at the same time point as the inferior lead electrocardiographs 100, 400, 500, and 600. For example, the connection event occurs every 20 ms during the measurement period for 30 seconds.

In order to implement the present invention, the following conditions are required: The inferior lead electrocardiographs 100, 400, 500, and 600 are required to transmit the constant number of sampling data in one connection event. Therefore, the sampling is not allowed to overlap the connection event in view of time. It is noted that the connection interval is required to be exactly an integer multiple of the sampling period to prevent the sampling and connection events from overlapping in view of time. In the embodiment of FIG. 7, six samplings are performed in one electrocardiogram during one connection interval. In addition, it is noted that the sampling period has the same value as T regardless of the presence of the connection event between two consecutive samplings.

It is very important in the present invention that the sampling and the Bluetooth Low Energy connection event do not overlap in view of time. The expression that the sampling and the connection event do not overlap in view of time signifies that the first sampling after the occurrence of the connection event is performed within a shorter time than the sampling period after the connection event starts. According to the present invention, the two electrocardiographs are required to be sampled at the same time point. In Bluetooth Low Energy, the master and the slave perform the connection event at the same time point. Therefore, the watch electrocardiograph 200 and the inferior lead electrocardiograph 100, 400, 500, and 600 perform sampling, respectively, when the same amount of time has elapsed since the start of the connection event. Then, the two electrocardiographs obtain two sampling values sampled at the same time (synchronously).

For example, in FIG. 7, the 6 samples sampled after one connection event is completed are stored in a temporary memory and transmitted at the immediately following connection event. The electrocardiograph receiving the transmitted 6 sampled data may sequentially substitute the 6 sampled data into Equations 7 to 10 or Equations 11 to 14 together with the 6 sampled data sampled by the electrocardiograph in the same time band. Then, for example, 4 leads*6 samples/lead=24 samples may be generated.

The example of transmitting data measured by the inferior lead electrocardiograph 100, 400, 500, and 600 to the watch electrocardiograph 200 has been described according to the present invention. However, it may be difficult to display the six electrocardiogram leads since the display of the watch is small. Accordingly, the two electrocardiogram lead data collected by the watch electrocardiograph 200 may be transmitted to a smartphone, and the smartphone may calculate four electrocardiogram lead signals and display the six electrocardiogram lead signals. Alternatively, at first, the two electrocardiographs may transmit the measured data to a smartphone and the smartphone may display 6 lead signals by calculating four electrocardiogram lead signals. In this case, a method equivalent to that of FIG. 7 may be used.

Hereinafter, when and why the electrocardiogram measurement start command (the command to start electrocardiogram measurement) occurs will be described according to the present invention. Arrhythmias may be intermittent and asymptomatic. Accordingly, a photoplethysmograph (PPG) may be mounted on a watch, so that a pulse or heart activity may be continuously monitored by using the photoplethysmograph. The photoplethysmograph has the advantage of performing measurements while being simply worn in one hand. When the PPG, which is monitoring cardiac activity, detects an abnormality in the cardiac activity, that is, when detects the symptom of arrhythmia, the PPG may generate an alarm. The alarm may be in the form of sound, vibration, or light. The user may measure the electrocardiogram after detecting the alarm. Particularly, in the present invention, two electrocardiographs may be used to measure two electrocardiogram lead signals. Therefore, when a predetermined amount of time elapses after the PPG generates the alarm, the watch may transmit an electrocardiogram measurement command to the inferior lead electrocardiograph 100, 400, 500, and 600.

Upon sensing the alarm, the user brings the opposite hand wearing the watch into contact with the corresponding electrode of the watch. Then, the current sensor of the watch detects the contact of the opposite hand, finishes preparing for the measurement of lead I, and attempts the connection of Bluetooth Low Energy. In addition, the user brings the corresponding electrode of the inferior lead electrocardiograph (watch electrocardiograph 100 and ring-shaped electrocardiograph 400) in contact with the left leg. Then, the current from the current sensor of the inferior lead electrocardiograph 100 and 400 flows between the left leg and the hand wearing the inferior lead electrocardiograph 100 and 400. Then, when the current sensor of the inferior lead electrocardiograph 100 and 400 detects the contact of the left leg and generates an output, the microcontroller of the inferior lead electrocardiograph 100 and 400 attempts the connection of Bluetooth Low Energy after preparing for electrocardiogram measurement. In addition, in the embodiment of the present invention, the microcontroller of the patch-shaped inferior lead electrocardiograph 500 and the chest band-shaped inferior lead electrocardiograph 600 may be activated by a scheme such as a mechanical switch in order to perform the electrocardiogram measurement according to the present invention. Then, the microcontroller may attempt the connection of Bluetooth Low Energy after finishing the preparation of electrocardiogram measurement suitable for the present invention.

When the Bluetooth Low Energy connection is established between the watch electrocardiograph 200 and the inferior lead electrocardiograph 100, 400, 500, and 600, the watch electrocardiograph 200 may transmit the electrocardiogram measurement command to the inferior lead electrocardiograph 100, 400, 500, and 600. According to the embodiments, the electrocardiogram measurement command may be transmitted by the inferior lead electrocardiographs 100, 400, 500, and 600 to the watch electrocardiograph 200.

When the user wants to measure the electrocardiogram even though the PPG of the watch does not generate the alarm, according to the principle of the present invention, i) the user may bring body parts into contact with corresponding to the two electrodes of the watch electrocardiograph 200 and the inferior lead electrocardiograph 100, 400, 500, and 600 or operate the mechanical switch or the like, and then ii) the two electrocardiographs may establish the connection of Bluetooth Low Energy, iii) one of the electrocardiographs may generate the electrocardiogram measurement command, and iv) the two electrocardiogram lead measurements described above may be performed.

The concept and principle of the present invention have been disclosed. The contents described in the embodiments of the present invention may be implemented more variously according to the concept and principle of the present invention.

The present invention has been described with the details such as specific elements, the limited embodiments, and the drawings, however, the above description is provided only to help a comprehensive understanding of the present invention, and the present invention is not limited to the embodiments. It will be understood by those skilled in the art that various changes and modifications may be carried out from the above-mentioned description.

Accordingly, the idea of the present invention will not be limited to the embodiments described above, and the following claims as well as all modifications or variations belonging to the equivalents of the claims will be within the scope of the invention.

1. A wearable device comprising:
a watch worn by a user on one wrist;
one band coupled to the watch;
a first electrocardiograph coupled to the one band and disposed at a position facing a bottom surface of the watch; and
a second electrocardiograph included in the watch, wherein
the first electrocardiograph includes a first electrode disposed on an inner surface of the band to come into contact with the one wrist of the user, and a second electrode disposed on an outer surface of the band to come into contact with a left knee or left ankle of the user, and
the second electrocardiograph includes a third electrode disposed on the bottom surface of the watch to come into contact with the one wrist of the user, and
a fourth electrode coming into contact with an opposite hand of the user.

2. The wearable device of claim 1, wherein
the first electrocardiograph
measures a first electrocardiogram lead signal induced between the first and second electrodes, and
transmits the measured first electrocardiogram lead signal to the second electrocardiograph by using a wireless communication mechanism, and
the second electrocardiograph
measures a second electrocardiogram lead signal through the third and fourth electrodes,
receives the first electrocardiogram lead signal by using the wireless communication mechanism, and
compensates for a time delay generated in a wireless communication process to the received first electrocardiogram lead signal so that the first electrocardiogram lead signal and the second electrocardiogram lead signal become two electrocardiogram lead signals sampled at the same time.

3. The wearable device of claim 2, wherein
the wearable device additionally calculates four electrocardiogram lead signals by using the two electrocardiogram lead signals sampled at the same time, thereby acquiring six limb lead signals including lead I, lead II, lead III, lead aVR, lead aVL and lead aVF.

4. The wearable device of claim 1, wherein the first electrocardiograph includes one microcontroller for controlling the first electrocardiograph, and the microcontroller is operated in a sleep mode when the first electrocardiograph does not measure an electrocardiogram lead signal to power off an amplifier, an AD (analog to digital) converter, and the wireless communication mechanism included in the first electrocardiograph, and powers on the amplifier, the AD converter and the wireless communication mechanism, when switched to an active mode, to amplify and AD convert the first electrocardiogram lead signal, and perform wireless communication.

5. The wearable device of claim 4, wherein the first electrocardiograph includes one current sensor supplied with power,
the current sensor, when the first electrode contacts the one wrist of the user and the second electrode contacts the left knee or the left ankle of the user, allows a current to flow through the user's body and generates an output signal upon sensing the current, and
the microcontroller changes a sleep mode to an active mode when receiving the output signal of the current sensor.

6. The wearable device of claim 2, wherein the wearable device uses a time delay value determined using the following processes (a) to (d):
(a) commonly applying one output signal of one signal generator to the first electrocardiograph and the second electrocardiograph;
(b) measuring, by the first electrocardiograph and the second electrocardiograph, the output signal;
(c) transmitting, by the first electrocardiograph, the measured signal through the wireless communication mechanism and receiving, by the second electrocardiograph, the transmitted signal; and
(d) comparing two waveforms of the signal measured by the second electrocardiograph and the signal received by the second electrocardiograph.

7. The wearable device of claim 1, wherein the band is configured such that a length of the band is formed longer than a length of the opposite band to accommodate the first electrocardiograph.

8. The wearable device of claim 2, wherein the wireless communication mechanism includes Bluetooth Low Energy.

9. A method for acquiring a plurality of electrocardiogram lead signals by using an electrocardiograph accommodated in a watch worn on one wrist and an electrocardiograph attached to a band of the watch, the method comprising:
bringing a first electrode of the electrocardiograph attached to the band into contact with a wrist and bringing a second electrode into contact with a left leg or a left ankle;
switching a microcontroller accommodated in the electrocardiograph attached to the band to an active mode;
powering on the amplifier, the AD converter, and the wireless communication mechanism when the microcontroller is switched to active mode;
amplifying an electrocardiogram lead signal between the first electrode and the second electrode;
converting the amplified analog signal into a digital signal;
transmitting the first electrocardiogram lead signal converted into the digital signal to an electrocardiograph accommodated in the watch by using the wireless communication mechanism;
receiving, by the electrocardiograph accommodated in the watch, the transmitted first electrocardiogram lead signal through the wireless communication mechanism; and
making the first electrocardiogram lead data and second electrocardiogram lead data which is measured through electrodes attached to the watch by compensating for a time delay to the received first electrocardiogram lead data, which is generated during a wireless communication process and the like, as a set of two electrocardiogram lead data sampled in a same time band.

10. The method of claim 9, further comprising:
checking a presence of a flow of a current in a current sensor to determine whether to terminate the electrocardiogram measurement, after the microcontroller accommodated in the electrocardiograph attached to the band measures the electrocardiogram for a predetermined period of time.

11. The wearable device of claim 3, wherein a difference between time points for sampling the two electrocardiogram lead signals is less than a sampling period to obtain the two electrocardiogram lead signals sampled in the same time band.

12. A wearable device comprising:
one watch electrocardiograph installed in one watch body to measure lead I; and
one inferior lead electrocardiograph for measuring one of lead II or lead III according to an installed position, wherein
the watch electrocardiograph wirelessly transmits a command for starting electrocardiogram measurement (electrocardiogram measurement start command) to the one inferior lead electrocardiograph,
the watch electrocardiograph measures lead I,
the one inferior lead electrocardiograph wirelessly receiving the electrocardiogram measurement start command measures one of lead II or lead III, and
the one inferior lead electrocardiograph wirelessly transmits the measured one of leads II or lead III to the watch electrocardiograph, so that the watch electrocardiograph wirelessly receives the transmitted one of the lead II or lead III, so as to acquire two electrocardiogram lead signals measured in a same time band; and
four electrocardiogram lead signals are additionally calculated by using the two electrocardiogram lead signals measured in the same time band, so as to acquire six limb lead signals including Lead I, Lead II, Lead III, Lead aVR, Lead aVL, and Lead aVF.

13. The wearable device of claim 12, wherein the one inferior lead electrocardiograph for measuring one of lead II or lead III includes one electrode coupled to one band coupled to the one watch body, disposed at a position facing the bottom surface of the watch body, and disposed on an inner surface of the band to contact the one wrist of the user, and one electrode disposed on an outer surface of the band to come into contact with a left knee or left ankle of the user.

14. The wearable device of claim 12, wherein the one inferior lead electrocardiograph for measuring one of lead II or lead III has a ring shape worn on one finger.

15. The wearable device of claim 12, wherein the one inferior lead electrocardiograph for measuring one of lead II or lead III has a patch or chest-band shape, and includes electrodes in contact with the chest.

16. The wearable device of claim 12, wherein the two electrocardiogram lead signals measured in the same time band have the same frequency response characteristics.

17. The wearable device of claim 12, wherein the two electrocardiogram lead signals measured in the same time band have the same gain characteristics.

18. The wearable device of claim 12, wherein the two electrocardiogram lead signals measured in the same time band have a maximum amplitude error within +/−5%.

19. The wearable device of claim 12, wherein the two electrocardiogram lead signals measured in the same time band are sampled at the same sampling rate.

20. The wearable device of claim 12, wherein a wireless type for communicating between the watch electrocardiograph and one inferior lead electrocardiograph includes Bluetooth Low Energy.

21. The wearable device of claim 12, wherein the one inferior lead electrocardiograph, after connection of the Bluetooth Low Energy is established, samples the electrocardiogram lead signal during one connection interval, and transmits the sampled data during one connection event following the sampling.

22. The wearable device of claim 21, wherein the connection interval is an integral multiple of a sampling period when the one inferior lead electrocardiograph samples one electrocardiogram lead signal.

23. The wearable device of claim 12, wherein the watch electrocardiograph and the one inferior lead electrocardiograph sample each electrocardiogram lead signal at the same time by sampling each electrocardiogram lead signal after the same amount of time has elapsed from the connection event.

24. The wearable device of claim 12, wherein an operation of additionally calculating the four electrocardiogram lead signals, or an operation of displaying six limb lead signals is performed on a smartphone.

25. The wearable device of claim 12, wherein the one electrocardiograph generates the electrocardiogram measurement start command, after a photoplethysmograph mounted together with the one electrocardiograph detects an abnormality in cardiac activity and generates an alarm.

26. The wearable device of claim 12, wherein the inferior lead electrocardiograph or the watch electrocardiograph generates the electrocardiogram measurement start command, after a current sensor detects that the user has brought the user's body into contact with two electrodes of the inferior lead electrocardiograph to measure the electrocardiogram and generates an output.
